(19) United States
(12) Patent Application Publication
(10) Pub. No.: US 2025/0295907 A1
(43) Pub. Date: Sep. 25, 2025

(54) Windkessel Simulation Apparatus

(71) Applicant: VentriFlo, Inc., Pelham, NH (US)

(72) Inventors: Douglas E. Vincent, Pelham, NH (US); David Olney, Chester, NH (US)

(21) Appl. No.: 18/613,864

(22) Filed: Mar. 22, 2024

(51) Int. Cl. A61M 60/117; A01N 1/02; A61M 60/405

(52) U.S. Cl. CPC A61M 60/117; A01N 1/143; A01N 1/148; A61M 60/405

(57) ABSTRACT

A Windkessel simulation apparatus includes a flexible fluid container that cyclically fills with a perfusate fluid, such as blood, and helps deliver the perfusate fluid to a heart, by means of an elastic reactive squeezing mechanism that applies compressive force proportional to the pressure in the flexible fluid container. The apparatus acts as a hydraulic capacitor, similarly to how arteries act in the body-inflating during systole, and squeezing during diastole. In this way, the apparatus may help simulate a natural circulatory system for an organ, such as an ex vivo heart.

### BACKGROUND

The Windkessel effect is the effect on arterial blood pressure due to the elastic compliance of arteries. Arteries fill with blood and distend during systole, and then recoil during diastole, effectively acting as a hydraulic capacitor, which dampens blood pressure fluctuation and supports perfusion of organs during diastole.

### SUMMARY

A Windkessel simulation apparatus comprises a flexible fluid container having an exterior, a top, a bottom, and a set of ports. The flexible fluid container is configured to receive fluid through at least one port of the set of ports and to deliver fluid to a heart through at least one port of the set of ports. The Windkessel simulation apparatus also comprises a reactive squeezing mechanism configured to receive the flexible fluid container and to apply compressive pressure to its exterior in response to an increase in fluid within the flexible fluid container, such that the compressive pressure causes fluid to be driven toward the heart through at least one port of the set of ports.

The reactive squeezing mechanism may comprise a back surface, a front plate, and a set of elastic members configured to compress the front plate and back surface together, such that the elastic members provide the compressive pressure when the flexible fluid container is disposed between the front plate and the back surface. The front plate may be pivotally and removable attached to the back surface proximate to a first edge of the front plate. The back surface may comprise a set of threaded hanging posts on which the flexible fluid container may be hung, and the set of elastic members may comprise a set of spring retention screws configured to engage with the threaded hanging posts. The front plate may be transparent and the back surface may be heated. The flexible fluid container may comprise an air elimination port oriented upwards such that it enables air to escape the flexible fluid container.

An ex vivo circulation system may have a flow path that includes the Windkessel simulation apparatus. The ex vivo circulation system may further comprise: an ex vivo box configured to hold an ex vivo heart having a right atrium, a right ventricle, a left atrium, and a left ventricle; a main collection reservoir; a pump; and an oxygenator. The ex vivo circulation system may have the heart in an unloaded state, where the pump is a pulsatile pump synchronized to the heart in a counter-pulse fashion, and fluid flows from the oxygenator to the Windkessel simulation apparatus. The ex vivo circulation system may further comprise a preload reservoir having a positive head height above the heart, and have the heart in a loaded state, where fluid flows from the oxygenator to the preload reservoir, and then to the left atrium of the heart. The ex vivo circulation system may further comprise a preload reservoir having a positive head height above the heart, and have the heart in a partially loaded state, where fluid flows from the oxygenator to the preload reservoir and then to the left atrium of the heart, and fluid also flows from the oxygenator to the Windkessel simulation apparatus.

A method of perfusing a heart comprises connecting a heart, having a right atrium, a right ventricle, a left atrium, and a left ventricle, to an ex vivo circulation system. The ex vivo circulation system has a Windkessel simulation apparatus comprising: a flexible fluid container having an exterior, a top, a bottom, and a set of ports; wherein the flexible fluid container is configured to receive fluid through at least one port of the set of ports and to deliver fluid to a heart through at least one port of the set of ports; a reactive squeezing mechanism configured to receive the flexible fluid container and to apply compressive pressure to its exterior in response to an increase in fluid within the flexible fluid container, such that the compressive pressure causes fluid to be driven toward the heart through at least one port of the set of ports. The ex vivo circulation system also comprises: an ex vivo box configured to hold the heart; a main collection reservoir; a pump; and an oxygenator. The method further comprises causing perfusion fluid to flow through the ex vivo circulation system.

The pump may be a pulsatile pump. The ex vivo circulation system may further comprise a preload reservoir having a positive head height above the heart, and be configured such that fluid flows from the oxygenator to the preload reservoir, and from the preload reservoir into the heart. Fluid may also flow from the oxygenator to the Windkessel simulation apparatus. The reactive squeezing mechanism may comprise a back surface, a front plate, and a set of elastic members configured to compress the front plate and back surface together, such that the elastic members provide the compressive pressure when the flexible fluid container is disposed between the front plate and the back surface. The front plate may be pivotally and removably attached to the back surface proximate to a first edge of the front plate. The back surface may comprise a set of threaded hanging posts on which the flexible fluid container may be hung, and the set of elastic members may comprise a set of spring retention screws configured to engage with the threaded hanging posts, and the front plate may comprise a set of holes configured to line up with the set of threaded hanging posts, the set of holes being disposed proximate to a second edge of the front plate, opposite the first edge of the front plate.

### BRIEF DESCRIPTION OF THE DRAWINGS

The foregoing features of embodiments will be more readily understood by reference to the following detailed description, taken with reference to the accompanying drawings, in which:

FIG. 1 is front view of a Windkessel simulation apparatus in accordance with an embodiment of the present invention;

FIG. 2 is a perspective view of the Windkessel simulation apparatus of FIG. 1, as seen from a position slightly above and to the side of the apparatus;

FIG. 3 is a side view of the Windkessel simulation apparatus of FIG. 1 with the hinge plate pivoted away from the bag of the apparatus;

FIG. 4 is a side view of the Windkessel simulation apparatus of FIG. 1 with the hinge plate pivoted against the bag of the apparatus with the spring retention screw in position;

FIG. 5 is a front view of the bag of the Windkessel simulation apparatus of FIG. 1;

FIG. 6 is an exploded view of the spring retention screw and related components of FIG. 4;

FIG. 7 is a flow schematic of the Windkessel simulation apparatus of FIG. 1 used ex vivo in a configuration wherein the heart is perfused but unloaded;

FIG. 8 is a flow schematic of the Windkessel simulation apparatus of FIG. 1 used ex vivo in a configuration wherein the heart is loaded; and

FIG. 9 is a flow schematic in which the flows of FIGS. 7 and 8 are superposed.

### DETAILED DESCRIPTION OF SPECIFIC EMBODIMENTS

An apparatus is described herein that is capable of simulating the Windkessel effect on an ex vivo heart. The Windkessel simulation apparatus provides the appropriate volume and pressure of perfusate to accommodate the physiologic metabolic range of the heart under a variety of loading conditions. Additionally, the apparatus reduces or prevents unwanted air infusion into the coronary artery.

The apparatus allows for adjustable compliance settings in order to mimic a range of naturally occurring elastic arterial compliance during extracorporeal perfusion of an isolated heart. The apparatus comprises a fluid containment bag disposed above the isolated heart such that gravity assists in the delivery of perfusate (perfusion liquid) to the heart while allowing air to rise to the top of the bag and away from the heart. The bag is additionally disposed within an elastically compressive container, for example, between at least two plates which are elastically connected, thus providing an elastic compressive force on the bag. Alternatively, the bag itself may be elastic, providing increasing force as it is filled with fluid. When the bag inflates with fluid (such as blood from the heart, or a mechanical pump), the pressure in the bag increases. When no additional fluid is entering the bag, the elastic compressive force delivers fluid back toward the heart, in decreasing pressures. The bag is connected to at least one tube that fluidly connects it to the isolated heart. Additional tubes may connect the bag to a pressure gauge, an air purging system, and/or an oxygenator. The bag, plates, and tubes may be transparent to enable visual monitoring of the fluid pathways and flow.

The apparatus may be connected to a circuit that may further include a container for the isolated heart, the container having ECG electrodes so that the contractions of the heart can be monitored, and if desired, can allow for synchronization (in co-pulsed or counter-pulsed fashion) with a mechanical pump. The heart may expel fluid that is allowed to flow to a collection reservoir, from which the mechanical pump can draw fluid to be delivered to an oxygenator. From the oxygenator, the fluid may be pumped to either (or both of) a preload reservoir or the bag. From the preload reservoir, the fluid flows back into the heart.

Definitions. As used in this description and the accompanying claims, the following terms shall have the meanings indicated, unless the context otherwise requires:

A “set” includes at least one member.

A “Windkessel simulation apparatus” is an apparatus that simulates ex vivo the dynamic blood flow and blood pressure in a mammal resulting from elastic compliance of the mammal's arterial tree in combination with an upstream pulsatile source and a downstream resistance.

A “reactive squeezing” mechanism is a mechanism configured to provide resistive compressive force proportional to the amount of pressure (and therefore, distention), of a container.

“Fluid” describes perfusate fluid, e.g., blood.

A heart in a “loaded” state means that the heart is cyclically contracting and the heart ejects a stroke volume of fluid at physiologic pressures sufficient (or greater) to supply the total perfusion needs of the heart.

A heart in a “partially loaded” state means that the heart is cyclically contracting and the heart ejects a stroke volume of fluid at flow rates and pressures insufficient to supply the total perfusion needs of the heart, but greater than a minimal amount.

A heart in an “unloaded” state means that the heart is cyclically contracting, but the heart is ejecting only a minimal amount of fluid at minimal pressures through force of the contractions, and therefore has a lower metabolic consumption than when the heart is in a loaded state. A Langendorff state is an example of an unloaded state.

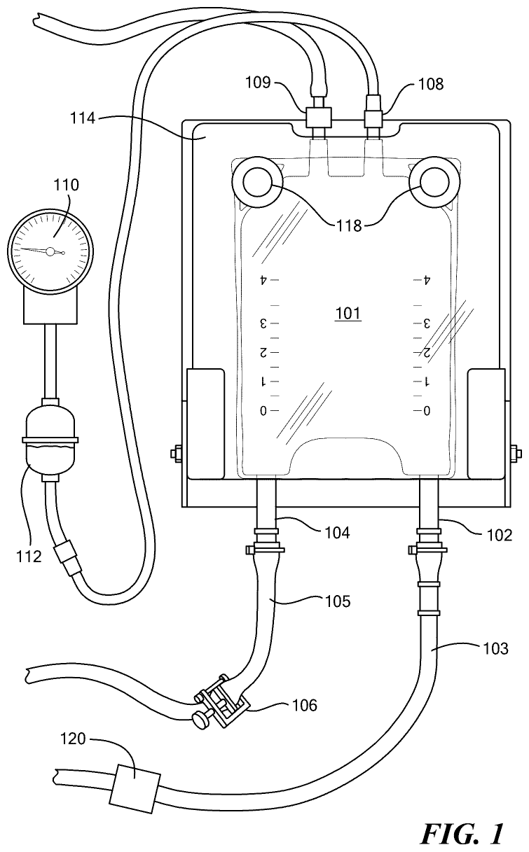

FIG. 1 is front view of a Windkessel simulation apparatus 100 (the “apparatus”) in accordance with an embodiment of the present invention. The apparatus 100 comprises a flexible fluid container 101, such as a blood bag, which can be filled with fluid, such as blood from a heart. The flexible fluid container 101 (the “bag”) is disposed between a back plate 202 (not clearly visible in FIG. 1) and a hinge plate 114 which are attached to each other, around the flexible fluid container 101, via, for example, a hinge and one or more spring retention screws 118. The spring retention screws 118 may provide adjustable elastic compression between the back plate 202 and the hinge plate 114. The bag 101 comprises at least one port. For example, the bag 101 may comprise a first port 102, which connects to a first tube 103 that further connects to a heart, for example to an aorta of the heart. The bag 101 may also comprise a second port 104, which may connect to a second tube 105 that further connects to an oxygenator, a mechanical pump, or a fluid reservoir. The second tube 105 may have a clamp 106, such as a Hoffman clamp on it, to selectively constrain or arrest fluid flow through the second tube. When the bag 101 fills with fluid pumped from the heart, or from a mechanical pump or fluid reservoir having a positive pressure, the pressure in the bag 101 increases. When the pressure in the bag 101 increases, the bag 101 delivers an outward force upon the hinge plate 114 and the back plate 202, which then, because of the elasticity of the spring retention screws, deliver an opposite compressive force upon the bag 101. This reactive squeezing functionality enables the apparatus 100 to simulate the Windkessel effect.

The bag 101 may further comprise a pressure gauge port 108, which connects (e.g., via a tube) to a pressure transducer protector 112, and further to a pressure gauge 110. The bag 101 may also comprise an air elimination port 109, which may be oriented upwards, such that it enables air to escape the bag 101. The bag 101 may be positioned above the heart such that gravity assists in driving fluid back into the heart, and so that air will rise toward the top of the bag 101 and away from the heart. The bag 101 may be positioned such that the first port 102 is oriented downwards, and/or toward the heart, such that gravity will assist in driving fluid back into the heart through the first port 102. The second port 104 may also be oriented downwards. The clamp 106 may be open when the heart is in an unloaded state, and closed or partially closed when the heart is in a loaded state. The first tube 103 may have a flow meter 120 to monitor flow between the heart and the bag 101. Other tubes and/or ports may optionally have flow meters as well.

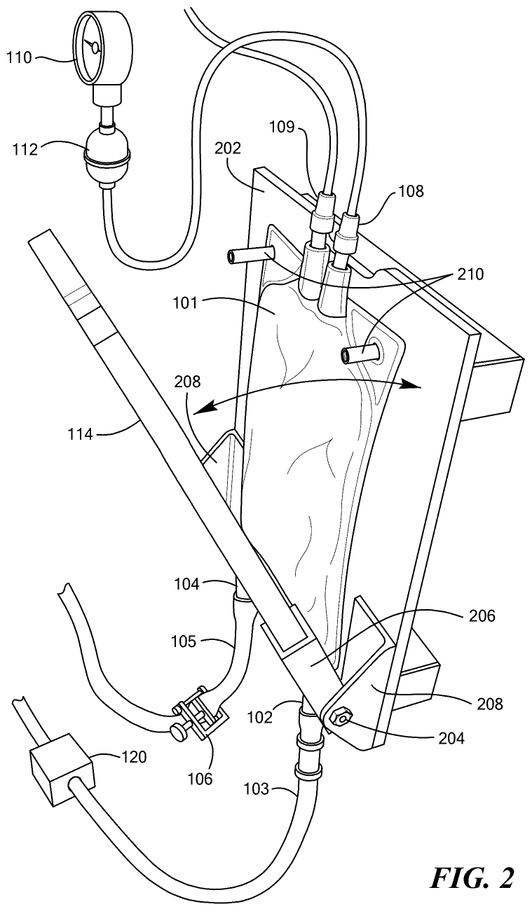

FIG. 2 is a perspective view of the Windkessel simulation apparatus 100 of FIG. 1, as seen from a position slightly above and to the side of the apparatus 100. In this figure, the hinge plate 114 is in an opened state, and is attached to the back plate 202 via a pivot 204, but not via spring retention screws 118. The hinge plate 114 may rotate about the pivot 204, the pivot 204 connecting a hinge plate receiver 206 to a hinge bracket 208. The hinge plate receiver 206 is configured to hold the hinge plate 114 and may have a U-shaped cross-section to enable the hinge plate 114 to be removed from the hinge plate receiver 206, when greater access to the bag 101 is desired. The hinge bracket 208 may be affixed to the back plate 202. The back plate 202 may further comprise threaded hanging posts 210, which may be used to hang the bag 101 (which may have holes that may be used to hang), and into (or onto) which the spring retention screws 118 may be screwed. When the hinge plate 114 is open, and the spring retention screws 118 are removed, the bag 101 may be removed from the back plate 202 for, e.g., cleaning or replacement.

There does not need to be a pivot 204—for example, a front plate could be elastically coupled at two or more points to a back plate with the bag 101 in between. The back plate 202 could be part of a wall, rather than a mounted plate. The hinge plate receiver 206 may have other cross section shapes, and/or may instead be permanently attached to the hinge plate 114. The hinge plate 114 could instead be directly attached to the pivot 204. The pivot 204, hinge plate receiver 206, or hinge bracket 208 may have a stop configured to constrain the range of motion of the hinge plate 114.

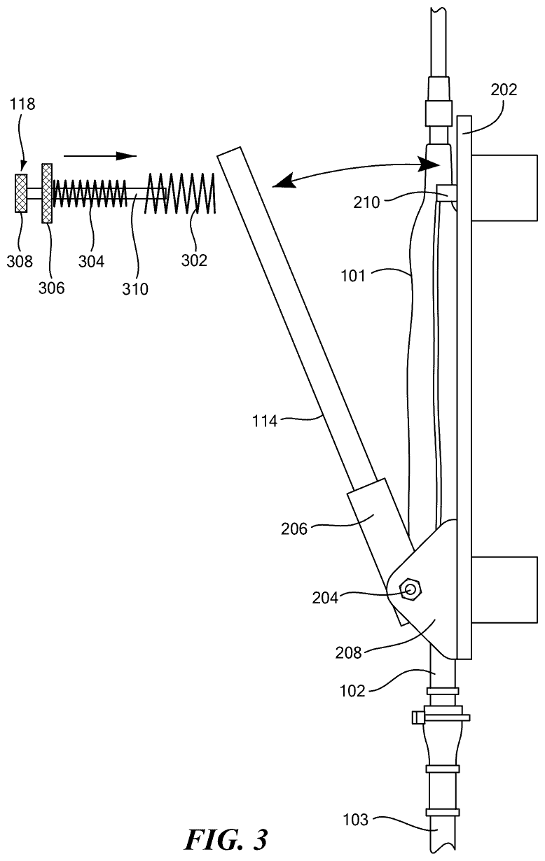

FIG. 3 is a side view of the Windkessel simulation apparatus 100 of FIG. 1 with the hinge plate 114 pivoted away from the bag 101 of the apparatus 100. In this view, a spring retention screw 118 is shown. The spring retention screw 118 may comprise an outer spring 302, an inner spring 304, a spring compression thumb wheel 306, a retention screw thumb wheel 308, and a retention screw shaft 310. The outer spring 302 may be disposed coaxially around the inner spring 304, which may further be disposed coaxially around the retention screw shaft 310. In some embodiments, there may be fewer or greater than two springs. The retention screw shaft 310 may be threaded (externally or internally) and configured to mate with a threaded hanging post 210. When mated, the spring retention screw 118 will retain the hinge plate 114 in a closed state. Other forms of mating other than threads may be used. The spring compression thumb wheel 306 may be used to adjust the level of compression in the springs 302 and 304, which will therefore adjust the level of elastic compliance in the apparatus 100. The elastic compliance may be adjusted such that the force exerted upon the bag 101 is within target parameters and such that fluid flows at a rate sufficient to perfuse the heart. When elastic resistance increases, the difference between the highest pressure and lowest pressure for a given pulsatile flow rate increases. When elastic resistance decreases, the difference between the highest pressure and lowest pressure for a given pulsatile flow rate decreases.

The retention screw thumb wheel 308 may be used to screw (or unscrew) the spring retention screw 118 into the threaded hanging post 210. The retention screw(s) 118 may be positioned perpendicularly to the back plate 202, substantially perpendicular (within 10 degrees of perpendicular), or at an angle with respect to the back plate 202. The hinge plate 114 may have holes through which the spring retention screw shafts 310 may be disposed, wherein the holes are sufficiently large to allow some movement of the hinge plate 114 in an arc, but not larger than the diameter of the inner spring 304. The spring compression thumb wheel 306 may have a diameter larger than the diameter of the outer spring 302.

Other forms of elastic compression may be used other than the spring retention screws 118. For example, springs or elastic bands may be positioned between (or around) the hinge plate 114 and the back plate 202. Elastic bands may be positioned around the hinge plate 114 and the bag 101. The bag 101 itself may have elastic properties, such that it applies compressive force to the fluid inside when inflated. There may be any number of spring retention screws 118, springs, elastic bands, or other methods of applying compressive force. The bag 101 may be positioned in such a way that a weight applies compressive force to the bag 101. Additionally, there does not need to be a pivot 204—for example, a front plate could be elastically coupled at three or more points to a back plate with the bag 101 in between. The back plate 202 could be part of a wall, rather than a mounted plate.

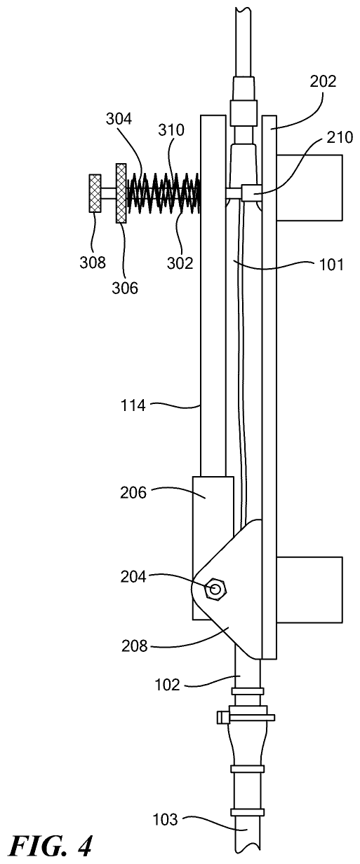

FIG. 4 is a side view of the Windkessel simulation apparatus 100 of FIG. 1 with the hinge plate 114 pivoted against the bag 101 of the apparatus 100 with the spring retention screw 118 in position. In this view, the hinge plate 114 is in a closed position, retained in such by one or more spring retainer screws 118. The springs 302 and 304 on the spring retainer screws 118 may be in a neutral or partially compressed state, providing compressive force upon the hinge plate 114 and thereby upon the bag 101 when the bag 101 inflates with fluid and thereby increases in pressure.

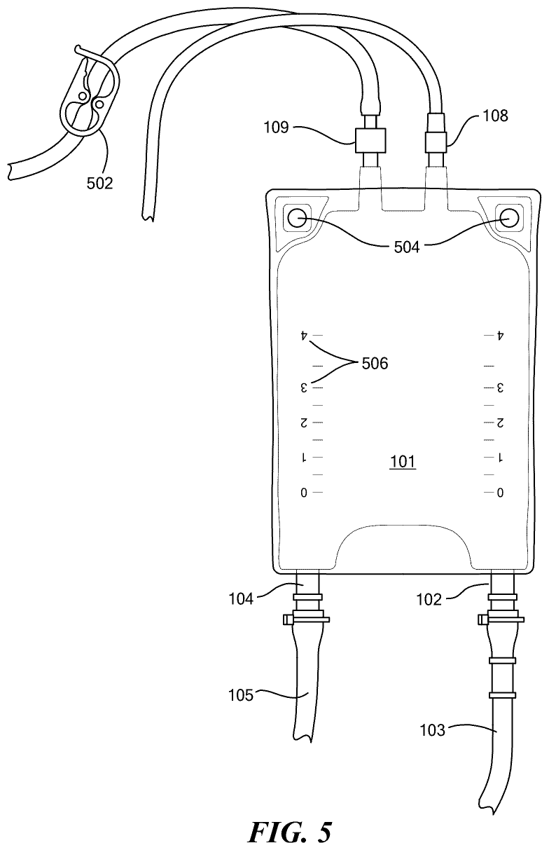

FIG. 5 is a front view of the bag 101 of the Windkessel simulation apparatus 100 of FIG. 1. The bag 101 may have a tube connected to an air elimination port 109, which may be clamped by a clamp 502, such as a Hoffman clamp, to control the rate of air purging from the bag 101 and/or to control the loss of volume (and therefore pressure) of fluid in the bag 101. The bag 101 may have one or more hanging holes 504, which allow the bag 101 to be hung by the threaded hanging posts 210. The bag 101 may have volume demarcations 506, which enable easy measurement of the volume of fluid contained in the bag 101. The bag 101 may instead be another form of hydraulic capacitor, such as a piston with adjustable resistance.

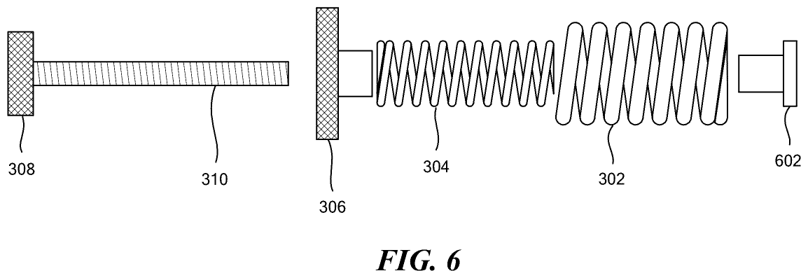

FIG. 6 is an exploded view of the spring retention screw 118 and related components of FIG. 4. In this view, there can be seen the retention screw thumb wheel 308 attached to the retention screw shaft 310. There is the spring compression thumb wheel 306, which may have a central threaded hole (not shown), through which the retention screw shaft 310 may be threaded, such that spring compression thumb wheel 306 may be moved along the retention screw shaft 310 by rotating the thumb wheel 306. When the spring compression thumb wheel 306 is moved along the retention screw shaft, i.e., toward the retention screw thumb wheel 308 or toward the hinge plate 114, the springs 302 and 304 are relaxed or compressed (between the spring compression thumb wheel 306 and the hinge plate 114), respectively. The spring retention screw 118 may further comprise a spring retention bushing 602. The spring retention bushing 602 may act to center the inner spring 304 and reduce interference between the springs 304 and 302.

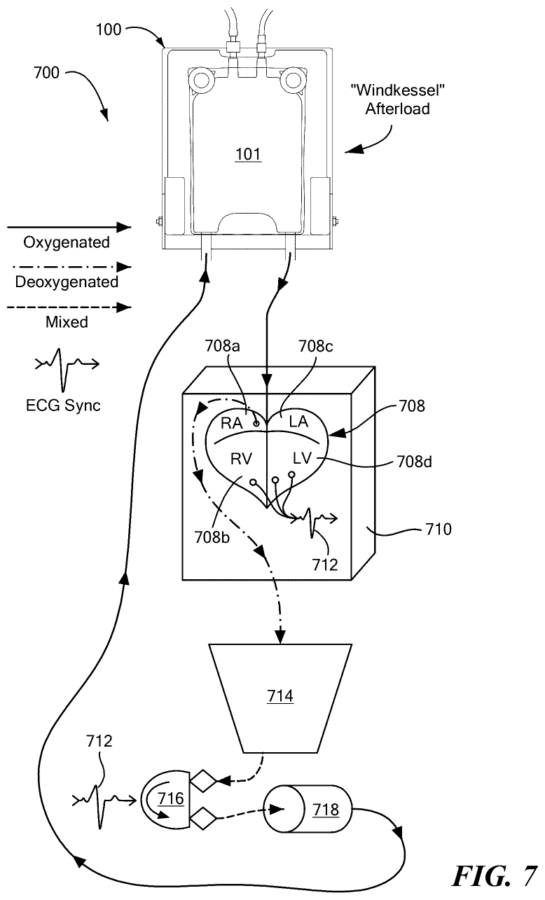

FIG. 7 is a flow schematic 700 of an ex vivo perfusion circuit utilizing the Windkessel simulation apparatus 100 of FIG. 1, in a configuration wherein the heart is perfused but unloaded. A heart may be inserted into an ex vivo perfusion circuit in an unloaded state in order to test whether the heart has basic contracting function, with minimal metabolic demand. In this embodiment, the Windkessel simulation apparatus 100 is connected via a first tube 103 to an ex vivo box 710, which may house an ex vivo heart 708 (the “heart”), which may be in an unloaded state. The heart 708 may comprise a right atrium 708a, a right ventricle 708b, a left atrium 708c, and a left ventricle 708d. Electrocardiogram (“ECG” a.k.a. “EKG”) electrodes may be connected to the heart 708 to measure the heart's 708 electrical activity in order to produce an ECG synchronization signal 712. In an unloaded state, the aorta is connected to a tube, which passes through the ex vivo box 710, which connects to the first tube 103. The left atrium 708c, the right atrium 708a, and the right ventricle 708b may be open to the atmosphere within the ex vivo box 710. The heart 708 may output fluid that flows into a main collection reservoir 714, having a negative head height relative to the heart 708, for example, through the ex vivo box 710.

Fluid within the main collection reservoir 714 may flow into a mechanical pump 716, such as a pulsatile pump (e.g., the pump according to U.S. patent application Ser. No. 17/183,080, the contents of which are incorporated herein by reference), which may be synchronized to the heart 708's beat in a counter-pulse fashion via the ECG synchronization signal 712. From the pump 716, the fluid may flow into an oxygenator 718, which oxygenates the fluid and may additionally comprise a heat exchanger to heat the fluid to a sub-normothermic temperature, e.g., 35° C. From the oxygenator 718, fluid may flow into the bag 101 of the Windkessel simulation apparatus 100, for example, through the second tube 105 and the second port 104.

Fluid flowing from the Windkessel apparatus 100 to the heart 708 may be oxygenated. Fluid flowing from the heart 708 to the main collection reservoir 714 may be deoxygenated, the heart 708 having consumed the oxygen in the fluid. There may be an air purge line (not shown) through which oxygenated (or partially oxygenated) fluid may flow from the air elimination port 109 to the main collection reservoir 714. Other lines not shown may flow into the main collection reservoir, such as fluid sampling lines. Fluid flowing from the main collection reservoir 714 to the pump 716 may then be partially oxygenated, which is then pumped into the oxygenator 718. Thereafter, fluid flowing from the oxygenator 718 to the apparatus 100 may be oxygenated and rewarmed.

To warm the perfusate and the system before the heart is connected, as well as to oxygenate the perfusate and purge air from the system, a resistance element is used in place of the heart and the system is run for a period of time.

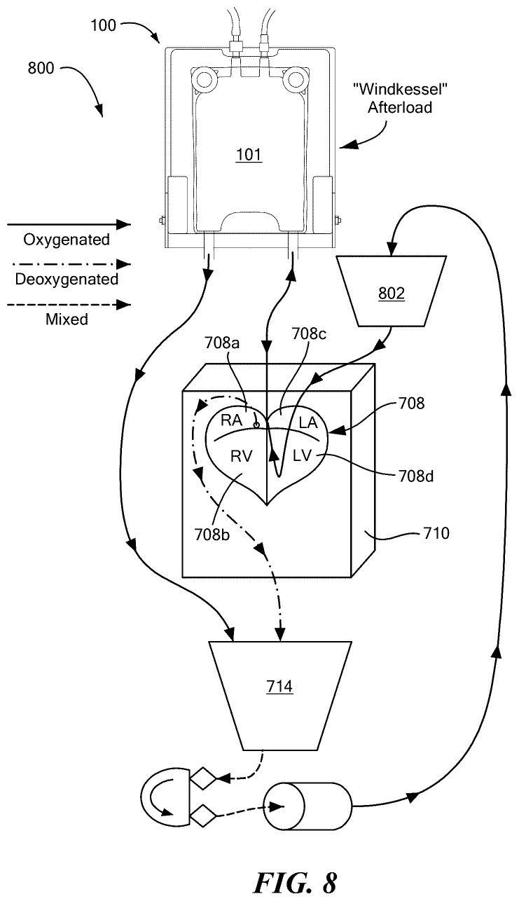

FIG. 8 is a flow schematic 800 of an ex vivo perfusion circuit utilizing the Windkessel simulation apparatus 100 of FIG. 1, in a configuration wherein the heart is perfused and loaded. A heart may be inserted into an ex vivo perfusion circuit in a loaded state in order to test whether the heart has sufficient (and strong enough) contracting function to be viable in a patient. In this embodiment, the Windkessel simulation apparatus 100 is connected via a first tube 103 to the ex vivo box 710, which may house the ex vivo heart 708, which may be in a loaded state. In the loaded state, the aorta is connected to a tube, which passes through the ex vivo box 710, which connects to the first tube 103. The left atrium 708c may be connected to a preload reservoir 802, while the right atrium 708a and the right ventricle 708b may be open to the atmosphere within the ex vivo box 710. The heart 708 may output fluid that flows into a main collection reservoir 714, having a negative head height relative to the heart 708, for example, through the ex vivo box 710.

Fluid within the main collection reservoir 714 may flow into a mechanical pump 716, such as a pulsatile pump (e.g., the pump according to U.S. patent application Ser. No. 17/183,080), which may be synchronized to the heart 708's beat via the ECG synchronization signal 712, or desynchronized from the heart 708's beat. From the pump 716, the fluid may flow into an oxygenator 718, which oxygenates the fluid and may additionally comprise a heat exchanger to heat the fluid to a sub-normothermic temperature, e.g., 35° C. From the oxygenator 718, fluid may flow into the preload reservoir 802, which is at a positive head height above the heart 708. From the preload reservoir 802, fluid may flow back into the heart 708, particularly into the left atrium 708c. The heart 708 may pump blood into the apparatus 100, which will increase the pressure in the bag 101, causing an outward force on the hinge plate 114 and the back plate 202, which will force the hinge plate 114 to compress the springs 302 and 304 of the spring retention screws 118. Once compressed, the springs 302 and 304 will exert a compressive force upon the hinge plate 114, causing the hinge plate to squeeze the bag 101. Thus, the apparatus 100 acts as a reactive squeezing mechanism upon the fluid in the bag 101, and provides physiologic (or other controllable target) pressure, timing, and flow to the heart 708's coronary arteries. From the apparatus 100, fluid may flow back into the heart 708 through the first tube 103, and/or may flow into the main collection reservoir 714 through the second port 104 and second tube 105, the air elimination port 109, or both. Fluid may be allowed to flow through the second port 104 and second tube 105 (controllable with an adjustable clamp) to provide pressure relief from the bag 101, in order to adjustably control the pressure in the bag 101, into which the heart 708 may pump when loaded. The pressure in the bag 101 may be tailored such that it remains within a target range and flows into the bag 101 and out of the bag 101 are matched.

Fluid flowing from the Windkessel apparatus 100 to the heart 708 may be oxygenated. Fluid flowing from the heart 708 to the main collection reservoir 714 may be deoxygenated, the heart 708 having consumed the oxygen in the fluid. There may be an air purge line (not shown) through which oxygenated (or partially oxygenated) fluid may flow from the air elimination port 109 to the main collection reservoir 714. Other lines not shown may flow into the main collection reservoir 714, such as fluid sampling lines. Fluid flowing from the apparatus 101 to the main collection reservoir 714 may be oxygenated. Fluid flowing from the main collection reservoir 714 to the pump 716 may then be partially oxygenated, which is then pumped into the oxygenator 718. Fluid flowing from the oxygenator 718 to the preload reservoir 802 may be oxygenated and rewarmed, resulting in fluid flowing from the preload reservoir 802 into the heart 708 being oxygenated and rewarmed (but may lose some heat due to resting in the preload reservoir 802 momentarily). Thereafter, the heart 708 may pump oxygenated blood into the apparatus 100.

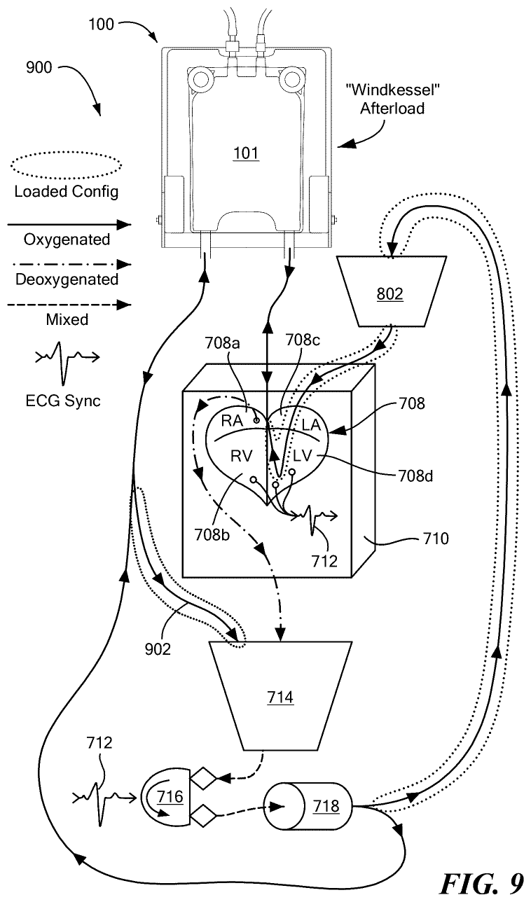

FIG. 9 is a full flow schematic 900 in which the flow circuits of FIGS. 7 and 8 are superimposed, which represents a realistic ex vivo perfusion circuit. In this figure, the elements, tubes, and connections shown and described in relation to FIGS. 7 and 8 (the unloaded and loaded configurations), are also shown. Flow may be directed through the tubes and/or connections encircled by dotted lines when the heart 708 is in a loaded state. Clamps or valves connected to the tubes/connections encircled by the dotted lines may be closed when the heart 708 is in an unloaded state, open when the heart 708 is in a loaded state, and may be some combination of open and closed (and/or in a partially closed state) when the heart 708 is in a partially loaded state. Thus, the heart 708 may be adjustably loaded. When the heart 708 is in an unloaded state, the flow path will resemble that shown in FIG. 7. When the heart 708 is in a loaded state, the flow path will resemble that shown in FIG. 8, and relief tube 902 will be open or partially open, depending on the strength of the heart 708. When the heart 708 is in a partially loaded state, the flow path will resemble that shown in FIG. 9, wherein some fluid flows from the oxygenator 718 to the preload reservoir 802, and then to the heart 708, and some fluid flows from the oxygenator 718 to the apparatus 100. Additionally, when the heart 708 is in a partially loaded state, relief tube 902 will be closed, e.g., via a valve or clamp.

There may be other lines not shown that enable flow into the main collection reservoir, such as fluid sampling lines and/or overflow prevention lines from the preload reservoir 802 and ex vivo box 710. There may be an air vent line connected to a tube disposed between the ex vivo box 710 and the main collection reservoir 714, configured to prevent fluid flow stoppage due to air bubbles. There may be a pressure gauge as shown in FIG. 1 connected to the bag 101. The ex vivo box 710 may be mounted on a pivot, such that the angle of the ex vivo box 710 may be adjusted to provide adjustable support to the heart 708. The ex vivo circuit as described is configured for manual control and monitoring, but may be automated with various controllers. The ex vivo circuit can be set up for laboratory experiment/monitoring, for clinical applications, or for transport to or from a hospital, or made transportable within a hospital or ambulance.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A Windkessel simulation apparatus comprising:
a flexible fluid container having an exterior, a top, a bottom, and a set of ports;
wherein the flexible fluid container is configured to receive fluid through at least one port of the set of ports and to deliver fluid to a heart through at least one port of the set of ports;
a reactive squeezing mechanism configured to receive the flexible fluid container and to apply compressive pressure to its exterior in response to an increase in fluid within the flexible fluid container,
such that the compressive pressure causes fluid to be driven toward the heart through at least one port of the set of ports.

2. A Windkessel simulation apparatus according to claim 1, wherein the reactive squeezing mechanism comprises a back surface, a front plate, and a set of elastic members configured to compress the front plate and back surface together, such that the elastic members provide the compressive pressure when the flexible fluid container is disposed between the front plate and the back surface.

3. A Windkessel simulation apparatus according to claim 2, wherein the front plate is pivotally and removably attached to the back surface proximate to a first edge of the front plate.

4. A Windkessel simulation apparatus according to claim 3, wherein the back surface comprises a set of threaded hanging posts on which the flexible fluid container may be hung, and wherein the set of elastic members comprise a set of spring retention screws configured to engage with the threaded hanging posts.

5. A Windkessel simulation apparatus according to claim 4, wherein the front plate comprises a set of holes configured to line up with the set of threaded hanging posts, and wherein the set of holes are disposed proximate to a second edge of the front plate, opposite the first edge of the front plate.

6. A Windkessel simulation apparatus according to claim 2, wherein the front plate is transparent.

7. A Windkessel simulation apparatus according to claim 2, wherein the back surface is heated.

8. A Windkessel simulation apparatus according to claim 1, wherein the flexible fluid container further comprises an air elimination port oriented upwards such that it enables air to escape the flexible fluid container.

9. An ex vivo circulation system having a flow path that includes the Windkessel simulation apparatus according to claim 1.

10. An ex vivo circulation system according to claim 9, further comprising: an ex vivo box configured to hold an ex vivo heart having a right atrium, a right ventricle, a left atrium, and a left ventricle; a main collection reservoir; a pump; and an oxygenator.

11. An ex vivo circulation system according to claim 10, wherein the heart is in an unloaded state, the pump is a pulsatile pump synchronized to the heart in a counter-pulse fashion, and fluid flows from the oxygenator to the Windkessel simulation apparatus.

12. An ex vivo circulation system according to claim 10, further comprising a preload reservoir having a positive head height above the heart, wherein the heart is in a loaded state, and fluid flows from the oxygenator to the preload reservoir and then to the left atrium of the heart.

13. An ex vivo circulation system according to claim 10, further comprising a preload reservoir having a positive head height above the heart, wherein the heart is in a partially loaded state, and fluid flows from the oxygenator to the preload reservoir and then to the left atrium of the heart, and fluid also flows from the oxygenator to the Windkessel simulation apparatus.

14. A method of perfusing a heart comprising:
(i) connecting a heart, having a right atrium, a right ventricle, a left atrium, and a left ventricle, to an ex vivo circulation system having:
a Windkessel simulation apparatus comprising:
a flexible fluid container having an exterior, a top, a bottom, and a set of ports;
wherein the flexible fluid container is configured to receive fluid through at least one port of the set of ports and to deliver fluid to a heart through at least one port of the set of ports;
a reactive squeezing mechanism configured to receive the flexible fluid container and to apply compressive pressure to its exterior in response to an increase in fluid within the flexible fluid container,
such that the compressive pressure causes fluid to be driven toward the heart through at least one port of the set of ports;
an ex vivo box configured to hold the heart;
a main collection reservoir;
a pump; and
an oxygenator;
(ii) causing perfusion fluid to flow through the ex vivo circulation system.

15. The method of claim 14, wherein the pump is a pulsatile pump.

16. The method of claim 14, wherein the ex vivo circulation system further comprises a preload reservoir having a positive head height above the heart, and wherein fluid flows from the oxygenator to the preload reservoir, and from the preload reservoir into the heart.

17. The method of claim 14, wherein fluid also flows from the oxygenator to the Windkessel simulation apparatus.

18. The method of claim 14, wherein the reactive squeezing mechanism comprises a back surface, a front plate, and a set of elastic members configured to compress the front plate and back surface together, such that the elastic members provide the compressive pressure when the flexible fluid container is disposed between the front plate and the back surface.

19. The method of claim 18, wherein the front plate is pivotally and removably attached to the back surface proximate to a first edge of the front plate.

20. The method of claim 19, wherein the back surface comprises a set of threaded hanging posts on which the flexible fluid container may be hung, and wherein the set of elastic members comprise a set of spring retention screws configured to engage with the threaded hanging posts, and wherein the front plate comprises a set of holes configured to line up with the set of threaded hanging posts, and wherein the set of holes are disposed proximate to a second edge of the front plate, opposite the first edge of the front plate.
